(19) United States
(12) Patent Application Publication
(10) Pub. No.: US 2023/0271932 A1
(43) Pub. Date: Aug. 31, 2023

(54) SMALL MOLECULE COVALENT ACTIVATORS OF UCP1

(71) Applicant: Dana-Farber Cancer Institute, Inc., Boston, MA (US)

(72) Inventors: Nathanael S. Gray, Boston, MA (US); Mengyang Fan, Allston, MA (US); Martha Ordonez, Brighton, MA (US); Edward Chouchani, Boston, MA (US); Tinghu Zhang, Brookline, MA (US); Zhengnian LI, Newton, MA (US); Jianwei Che, Sharon, MA (US)

(21) Appl. No.: 18/010,864

(22) PCT Filed: Jun. 16, 2021
(86) PCT No.: PCT/US21/37566

§ 371 Date: Dec. 16, 2022

Related U.S. Application Data
(60) Provisional application No. 63/039,643, filed on Jun. 16, 2020.

(51) Int. Cl. C07D 333/38; A61P 3/04; C07D 409/04; C07D 277/56; C07D 417/04

(52) U.S. Cl. CPC C07D 333/38; A61P 3/04; C07D 409/04; C07D 277/56; C07D 417/04

(57) ABSTRACT

Disclosed herein are compounds of Formula (I) and pharmaceutically acceptable salts thereof. The compounds of Formula (I) are useful for activating uncoupling protein 1 (UCP1) dependent thermogenesis. Also disclosed herein are methods of treating obesity or metabolic disorders such as diabetes using a compound of Formula (I).

### RELATED APPLICATION

This application claims priority to U.S. Provisional Patent Application Ser. No. 63/039,643, filed Jun. 16, 2020, the contents of which are hereby incorporated by reference.

### BACKGROUND

Activation of UCP1 stimulates calorie burning in pre-clinical and human studies. To date, only physiological stimuli (e.g. exposure to cold temperatures) have been shown to activate this protein. Even though UCP1 is essential for calorie burning in brown adipocytes, no small molecules have yet been shown to engage this target and affect activity.

There exists a need to develop small molecule activators of UCP1 that will be useful for stimulating calorie burning and as therapeutics for treating obesity and metabolic disorders.

### SUMMARY OF THE INVENTION

In certain aspects, the present disclosure provides compounds of Formula (I), and pharmaceutically acceptable salts thereof:

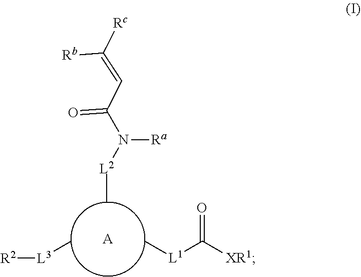

wherein:

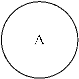

represents a heteroaryl or aryl ring;
L¹ represents —CH₂— or a bond;
L² represents —CH₂— or a bond;
R^{a} represents H or alkyl;
R^{b} represents H, alkyl, —C(O)OH, or —C(O)NH₂;
R^{c} represents H, alkyl, —C(O)OH, or —C(O)NH₂;
X represents —O—, —NH—, or —N(alkyl)-;
R¹ represents H or optionally substituted alkyl, cycloalkyl, aryl, or heteroaryl; or, XR¹, taken together, represent optionally substituted heterocycloalkyl, wherein the optionally substituted heterocycloalkyl is attached to the carbonyl group through a nitrogen atom;
L³ represents —NH— or a bond; and
R² represents optionally substituted aryl, heteroaryl, or heterocycloalkenyl.

The invention also provides pharmaceutical compositions comprising the compound of Formula (I).

In certain aspects, the present disclosure provides methods of activating uncoupling protein 1 (UCP1) in a cell comprising contacting the cell with a compound of Formula (I).

The present disclosure also provides methods of treating obesity, comprising administering to a subject a therapeutically effective amount of a compound of Formula (I).

The present disclosure also provides methods of lowering the weight of a subject, comprising administering to the subject a therapeutically effective amount of a compound of Formula (I).

The present disclosure also provides methods of stimulating calorie burning in a subject, comprising administering to the subject a therapeutically effective amount of a compound of Formula (I).

The present disclosure also provides methods of treating a metabolic disorder such as diabetes or nonalcoholic steatohepatitis, comprising administering to a subject a therapeutically effective amount of a compound of Formula (I).

### BRIEF DESCRIPTION OF THE DRAWINGS

FIG. 1A shows activation of UCP-1-dependent respiration in wild-type primary brown adipocytes with MYF-03-53.

FIG. 1B shows activation of UCP-1-dependent respiration in wild-type primary brown adipocytes with MYF-03-37.

FIG. 1C shows activation of UCP-1-dependent respiration in wild-type primary brown adipocytes with MYF-03-38.

FIG. 1D shows activation of UCP-1-dependent respiration in wild-type primary brown adipocytes with MYF-03-61.

FIG. 2A shows activation of UCP-1-dependent respiration in wild-type primary brown adipocytes with ZNL-06-030.

FIG. 2B shows activation of UCP-1-dependent respiration in wild-type primary brown adipocytes with ZNL-06-123.

FIG. 3A shows activation of UCP-1-dependent respiration in wild-type primary brown adipocytes with CP-A16.

FIG. 3B shows activation of UCP-1-dependent respiration in wild-type primary brown adipocytes with ZNL-06-058.

FIG. 4 shows activation of UCP-1-dependent respiration in UCP-1 knockout primary brown adipocytes with MYF-03-53.

### DETAILED DESCRIPTION OF THE INVENTION

Endogenous modification of UCP1 cysteine-253 disrupts the inactive conformation of this protein and drives therapeutic activation (Nature; 2016 Apr. 7; 532(7597): 112-6. doi: 10.1038/nature17399). The present disclosure provides a series of small molecules that potently activate UCP1-dependent calorie burning in brown adipocytes, and have no apparent stimulatory effects in non-adipocyte cells that lack UCP1. The compounds disclosed herein are the first-in-class covalent activators of UCP1-dependent thermogenesis.

In certain aspects, the present disclosure provides compounds of Formula (I), and pharmaceutically acceptable salts thereof:

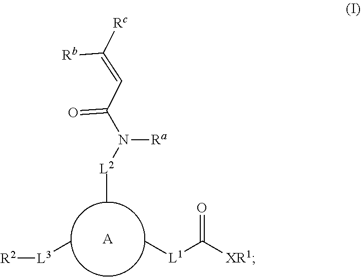

wherein:

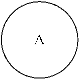

represents a heteroaryl or aryl ring;
L¹ represents —CH₂— or a bond;
L² represents —CH₂— or a bond;
R^{a} represents H or alkyl;
R^{b} represents H, alkyl, —C(O)OH, or —C(O)NH₂;
R^{c} represents H, alkyl, —C(O)OH, or —C(O)NH₂;
X represents —O—, —NH—, or —N(alkyl)-;
R¹ represents H or optionally substituted alkyl, cycloalkyl, aryl, or heteroaryl; or, XR¹, taken together, represent optionally substituted heterocycloalkyl, wherein the optionally substituted heterocycloalkyl is attached to the carbonyl group through a nitrogen atom;
L³ represents —NH— or a bond; and
R² represents optionally substituted aryl, heteroaryl, or heterocycloalkenyl.

In certain embodiments,

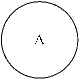

represents a heteroaryl ring, e.g., a pyrrole ring, a furan ring, an imidazole ring, a pyrazole ring, an oxazole ring, an isoxazole ring, an isothiazole ring, a triazole ring, an oxadiazole ring, a thiadiazole ring, a dithiazole ring, a tetrazole ring, a pyridine ring, a pyridazine ring, a pyrimidine ring, a pyrazine ring, a thiazole ring, or a thiophene ring. In certain embodiments,

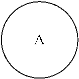

represents a 5-membered heteroaryl ring, e.g., a pyrrole ring, a furan ring, an imidazole ring, a pyrazole ring, an oxazole ring, an isoxazole ring, an isothiazole ring, a triazole ring, an oxadiazole ring, a thiadiazole ring, a dithiazole ring, a tetrazole ring, a thiazole ring, or a thiophene ring. In certain embodiments,

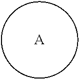

represents a 5-membered heteroaryl ring containing a sulfur atom, e.g., an isothiazole ring, a thiadiazole ring, a dithiazole ring, a thiazole ring, or a thiophene ring. In certain embodiments,

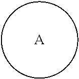

represents a 5-membered heteroaryl ring, e.g., a pyrrole ring, a furan ring, an imidazole ring, a pyrazole ring, an oxazole ring, an isoxazole ring, an isothiazole ring, a thiazole ring, or a thiophene ring. In certain preferred embodiments,

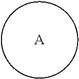

represents a thiazole ring or a thiophene ring. In certain embodiments,

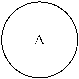

represents a thiophene ring.
In certain embodiments, the compound has the structure of formula (Ib):

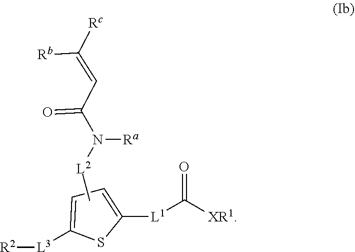

For example, the compound may have the structure of formula (Ic):

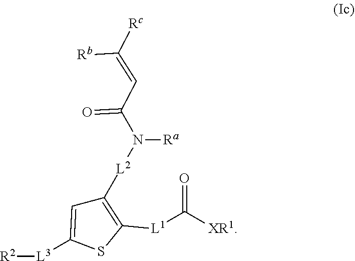

In other embodiments,

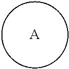

represents a thiazole ring.
For example, the compound may have the structure of formula (Id):

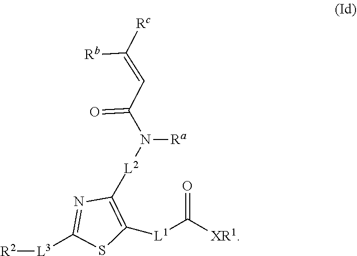

In other embodiments,

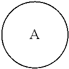

represents a thiazole ring.
For example, the compound may have the structure of formula (Id):

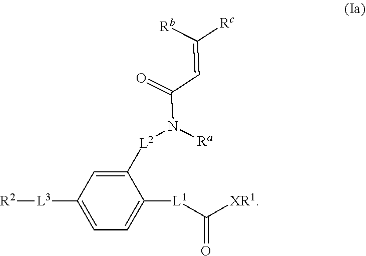

In certain embodiments, L¹ represents —CH₂—. Alternatively, L¹ may represent a bond.

In certain embodiments, L² represents —CH₂—. Alternatively, L² may represent a bond.

In certain embodiments, R^{a} represents H. Alternatively, R^{a} may represent alkyl, e.g., methyl.

In certain embodiments, X represents —O—. Alternatively, X represents —NH—.

In certain embodiments, R¹ represents H. In alternative embodiments, R¹ represents optionally substituted alkyl, e.g., methyl. In further embodiments, R¹ represents optionally substituted cycloalkyl, e.g., cyclopropyl. In still further embodiments, R¹ represents optionally substituted aryl, e.g., phenyl. In preferred embodiments, R¹ represents optionally substituted alkyl.

In certain embodiments, XR¹ represents —O(alkyl), —OH, —NH(alkyl), —NH(aryl), or —NH(cycloalkyl).

In certain embodiments, XR¹ represents —OCH₃, —OH, —NHCH₃, —NH(phenyl), or —NH(cyclopropyl).

In certain embodiments, XR¹, taken together, represents optionally substituted heterocycloalkyl, wherein the optionally substituted heterocycloalkyl is attached to the carbonyl group through a nitrogen atom. For example, XR¹ may be

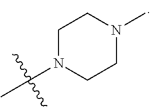 [SMILES: *N1CCN(C)CC1]

In certain embodiments, R² is optionally substituted aryl or heteroaryl.

In certain embodiments, R² is optionally substituted aryl. For example, R² may be optionally substituted phenyl or naphthyl.

For example, R² may be phenyl, optionally substituted with one or more substituents selected from halo, alkyl, haloalkyl, alkoxy, haloalkoxy, cyano, nitro, and (alkyl)sulfonyl.

In other preferred embodiments, R² is optionally substituted heteroaryl. For example, R² may be optionally substituted pyridyl, pyridazinyl, pyrimidinyl, pyrazinyl, pyrrolyl, furanyl, imidazolyl, pyrazolyl, oxazolyl, isoxazolyl, isothiazolyl, triazolyl, oxadiazolyl, thiadiazolyl, dithiazolyl, tetrazolyl, thiazolyl, or a thiophenyl.

For example, R² may be pyridyl, pyridazinyl, pyrimidinyl, or pyrazinyl, optionally substituted with one or more substituents selected from the group consisting of halo, alkyl, haloalkyl, alkoxy, haloalkoxy, cyano, nitro, and (alkyl)sulfonyl.

In certain embodiments, L³ represents a bond.

In certain embodiments, R^{b} represents H. Alternatively, R^{b} may represent —C(O)OH.

Alternatively still, R^{b} may represent —C(O)NH₂.

In certain embodiments, R^{c} represents H.

In certain embodiments, the compound of the invention has the structure of formula (Ie):

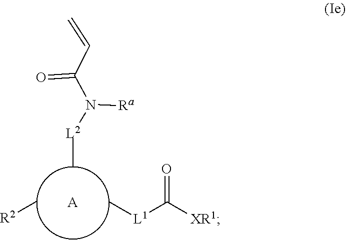

wherein:

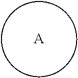

represents a heteroaryl or aryl ring;
L¹ represents —CH₂— or a bond;
L² represents —CH₂— or a bond;
R^{a} represents H or alkyl;
X represents —O—, —NH—, or —N(alkyl)-;
R¹ represents H or optionally substituted alkyl, cycloalkyl, aryl, or heteroaryl; and
R² represents optionally substituted aryl, heteroaryl.

In certain embodiments, the compound is selected from Table 1:

TABLE 1
Exemplary Compounds of the Present Disclosure

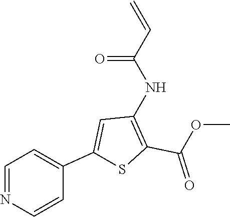 [SMILES: C=CC(=O)Nc1cc(-c2ccncc2)sc1C(=O)OC]

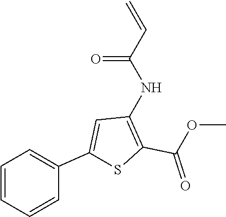 [SMILES: C=CC(=O)Nc1cc(-c2ccccc2)sc1C(=O)OC]

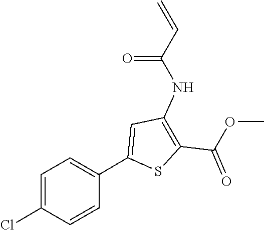 [SMILES: C=CC(=O)Nc1cc(-c2ccc(Cl)cc2)sc1C(=O)OC]

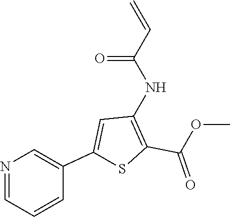 [SMILES: C=CC(=O)Nc1cc(-c2cccnc2)sc1C(=O)OC]

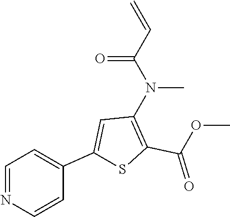 [SMILES: C=CC(=O)N(C)c1cc(-c2ccncc2)sc1C(=O)OC]

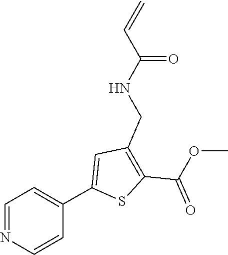 [SMILES: C=CC(=O)NCc1cc(-c2ccncc2)sc1C(=O)OC]

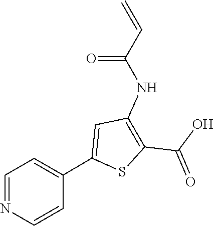 [SMILES: C=CC(=O)Nc1cc(-c2ccncc2)sc1C(=O)O]

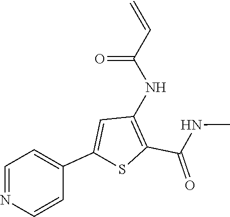 [SMILES: C=CC(=O)Nc1cc(-c2ccncc2)sc1C(=O)NC]

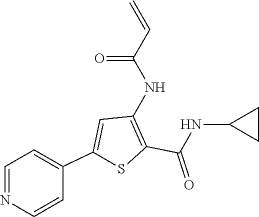 [SMILES: C=CC(=O)Nc1cc(-c2ccncc2)sc1C(=O)NC1CC1]

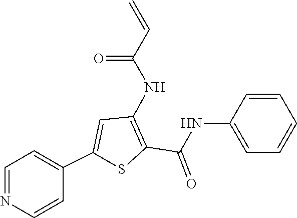 [SMILES: C=CC(=O)Nc1cc(-c2ccncc2)sc1C(=O)Nc1ccccc1]

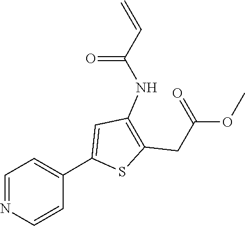 [SMILES: C=CC(=O)Nc1cc(-c2ccncc2)sc1CC(=O)OC]

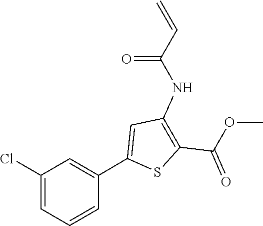 [SMILES: C=CC(=O)Nc1cc(-c2cccc(Cl)c2)sc1C(=O)OC]

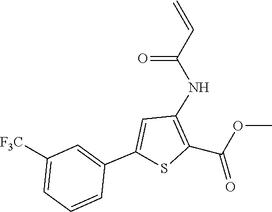 [SMILES: C=CC(=O)Nc1cc(-c2cccc(C(F)(F)F)c2)sc1C(=O)OC]

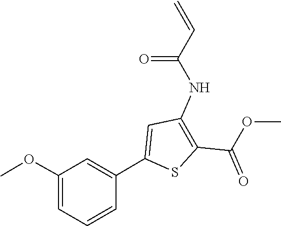

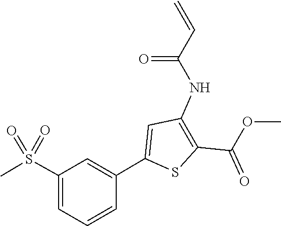

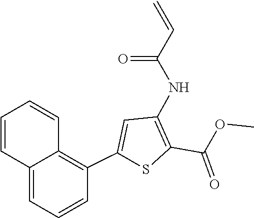 [SMILES: C=CC(=O)Nc1cc(-c2cccc3ccccc23)sc1C(=O)OC]

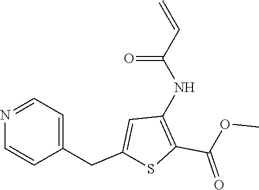 [SMILES: C=CC(=O)Nc1cc(Cc2ccncc2)sc1C(=O)OC]

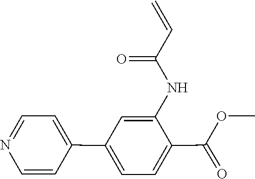 [SMILES: C=CC(=O)Nc1cc(-c2ccncc2)ccc1C(=O)OC]

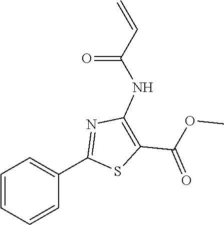 [SMILES: C=CC(=O)Nc1nc(-c2ccccc2)sc1C(=O)OC]

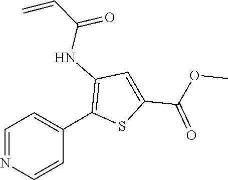 [SMILES: C=CC(=O)Nc1cc(C(=O)OC)sc1-c1ccncc1]

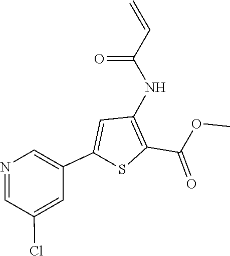 [SMILES: C=CC(=O)Nc1cc(-c2cncc(Cl)c2)sc1C(=O)OC]

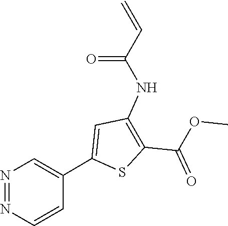 [SMILES: C=CC(=O)Nc1cc(-c2ccnnc2)sc1C(=O)OC]

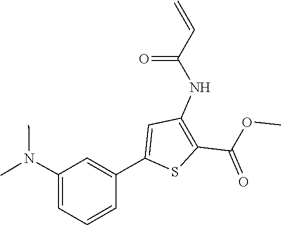

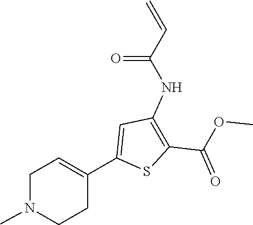

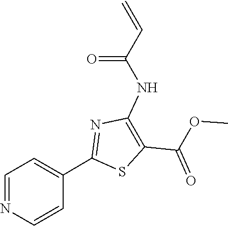 [SMILES: C=CC(=O)Nc1nc(-c2ccncc2)sc1C(=O)OC]

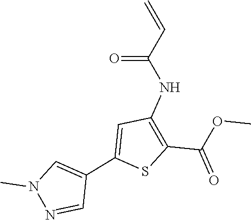

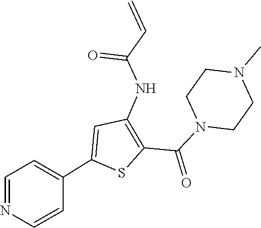 [SMILES: C=CC(=O)Nc1cc(-c2ccncc2)sc1C(=O)N1CCN(C)CC1]

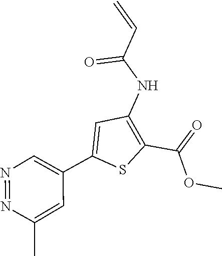 [SMILES: C=CC(=O)Nc1cc(-c2cnnc(C)c2)sc1C(=O)OC]

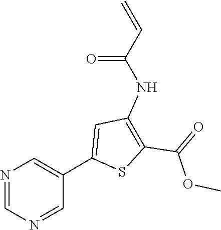 [SMILES: C=CC(=O)Nc1cc(-c2cncnc2)sc1C(=O)OC]

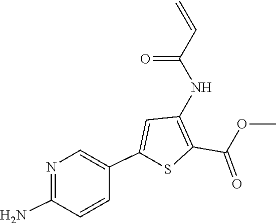 [SMILES: C=CC(=O)Nc1cc(-c2ccc(N)nc2)sc1C(=O)OC]

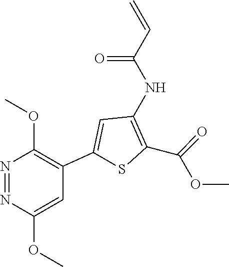 [SMILES: C=CC(=O)Nc1cc(-c2cc(OC)nnc2OC)sc1C(=O)OC]

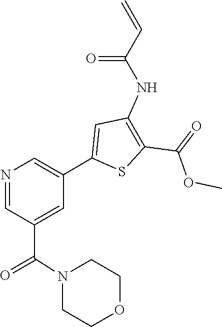 [SMILES: C=CC(=O)Nc1cc(-c2cncc(C(=O)N3CCOCC3)c2)sc1C(=O)OC]

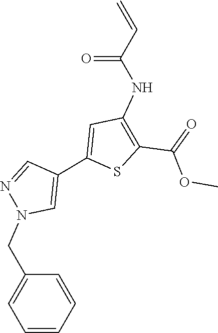 [SMILES: C=CC(=O)Nc1cc(-c2cnn(Cc3ccccc3)c2)sc1C(=O)OC]

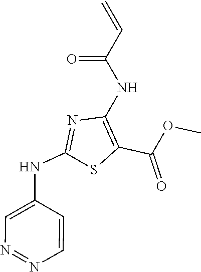

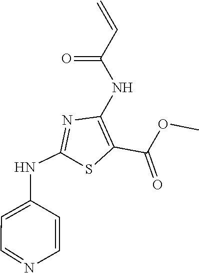

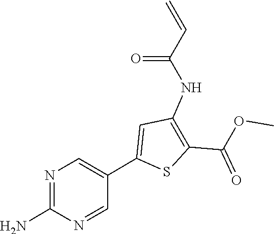 [SMILES: C=CC(=O)Nc1cc(-c2cnc(N)nc2)sc1C(=O)OC]

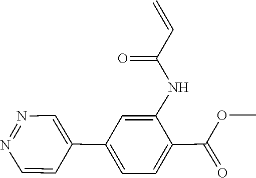

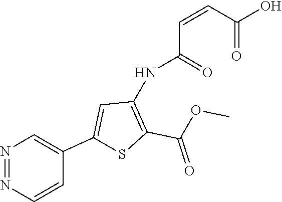 [SMILES: COC(=O)c1sc(-c2ccnnc2)cc1NC(=O)/C=C\C(=O)O]

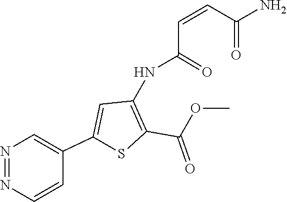 [SMILES: COC(=O)c1sc(-c2ccnnc2)cc1NC(=O)/C=C\C(N)=O]

In certain embodiments, the compound is a pharmaceutically acceptable salt of a compound of Table 1.

In certain aspects, the present disclosure provides a pharmaceutical composition comprising a compound of Formula (I) and at least one pharmaceutically acceptable excipient.

In certain embodiments, the invention provides a compound, or a pharmaceutically acceptable salt thereof, selected from the following table:

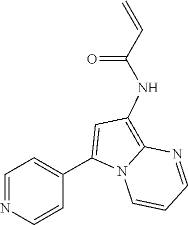 [SMILES: C=CC(=O)Nc1cc(-c2ccncc2)n2cccnc12]

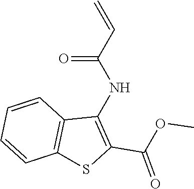 [SMILES: C=CC(=O)Nc1c(C(=O)OC)sc2ccccc12]

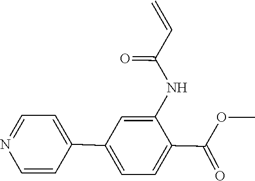 [SMILES: C=CC(=O)Nc1cc(-c2ccncc2)ccc1C(=O)OC]

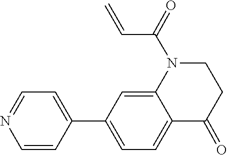 [SMILES: C=CC(=O)N1CCC(=O)c2ccc(-c3ccncc3)cc21]

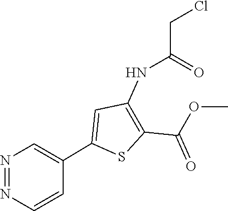 [SMILES: COC(=O)c1sc(-c2ccnnc2)cc1NC(=O)CCl]

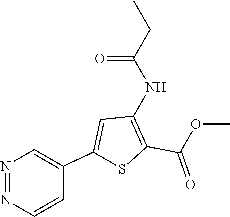 [SMILES: CCC(=O)Nc1cc(-c2ccnnc2)sc1C(=O)OC]

ZNL-06-123

### Methods of the Invention

In certain embodiments, the present disclosure provides methods of activating uncoupling protein 1 (UCP1) in a cell, comprising contacting the cell with a compound of Formula (I) or a composition thereof.

In certain embodiments, the method of activating UCP1 occurs in vitro. In alternative embodiments, the method occurs in vivo.

In certain embodiments, the present disclosure provides a method of treating obesity, comprising administering to a subject a therapeutically effective amount of a compound of Formula (I) or a pharmaceutical composition thereof.

In certain embodiments, the present disclosure provides a method of lowering the weight of a subject, comprising administering to the subject a therapeutically effective amount of a compound of Formula (I) or a pharmaceutical composition thereof.

In certain embodiments, the present disclosure provides a method of stimulating calorie burning in a subject, comprising administering to the subject a therapeutically effective amount of a compound of Formula (I) or a pharmaceutical composition thereof.

In certain embodiments, the present disclosure provides a method of treating a metabolic disorder, comprising administering to a subject a therapeutically effective amount of a compound of Formula (I) or a pharmaceutical composition thereof.

In certain embodiments, the metabolic disorder is diabetes. Alternatively, the metabolic disorder may be nonalcoholic steatohepatitis.

### Pharmaceutical Compositions

In certain embodiments, the disclosure provides a pharmaceutical composition comprising a compound of Formula (I),or a pharmaceutically acceptable salt thereof, and at least one pharmaceutically acceptable excipient.

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, l-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, Mass. (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, California (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result in chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, formyl, carboxyl, alkoxycarbonyl, thioester, thioacetate, thioformate, aryl, arylalkyl, heteroaryl, cycloalkyl, heterocyclyl, amino, aminoalkyl, amido, amidine, imine, cyano, azido, haloalkyl, haloalkoxy, sulfhydryl, an alkylthio, a sulfate, a sulfonate, a sulfamoyl, a sulfonamido, a sulfonyl, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆ alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

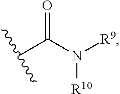

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

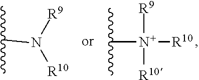

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl” or “arylalkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

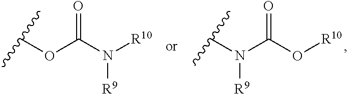

wherein R⁹ and R¹⁰ independently represent hydrogen or a hydrocarbyl group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “cycloalkyl” means mono- or bicyclic or bridged saturated carbocyclic rings, each having from 3 to 12 carbon atoms. Certain cycloalkyls have from 5-12 carbon atoms in their ring structure, and may have 6-10 carbons in the ring structure. Preferably, cycloalkyl is (C₃-C₇)cycloalkyl, which represents a monocyclic saturated carbocyclic ring, having from 3 to 7 carbon atoms. Examples of monocyclic cycloalkyls include cyclopropyl, cyclobutyl, cyclopentyl, cyclopentenyl, cyclohexyl, cyclohexenyl, cycloheptyl, and cyclooctyl. Bicyclic cycloalkyl ring systems include bridged monocyclic rings and fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl” and “heteroarylalkyl”, as used herein, refers to an alkyl group substituted with a heteroaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, “heterocycloalkyl,” and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heterocycloalkenyl” as used herein means a radical of a non-aromatic ring system, including, but not limited to, monocyclic, bicyclic, and tricyclic rings, having 3 to 12 atoms including at least one heteroatom, such as nitrogen, oxygen, or sulfur, and which contain at least one carbon-carbon double bond formed by the removal of two hydrogens. Representative examples of heterocycloalkenyl include 1,2,3,6-tetrahydropyridine.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms.

Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

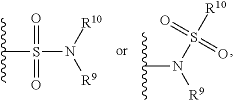

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

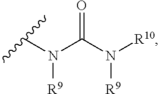

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Compounds of the disclosure may have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, compounds of the disclosure which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The disclosure now being generally set forth, it will be more readily understood by reference to the following examples which are included merely for purposes of illustration of certain aspects and embodiments of the present disclosure, and are not intended as limiting.

### Example 1: Preparation of Exemplary Compounds

Although specific procedures for exemplary compounds are provided below, the compounds of the invention may generally be made in a manner analogous to the General Synthetic Scheme I set forth below, and to the specific procedures set forth herein. For example, routine halogenation, cross coupling, and amidation reactions known in the art may be used to furnish compounds of the invention by analogy to the methods below.

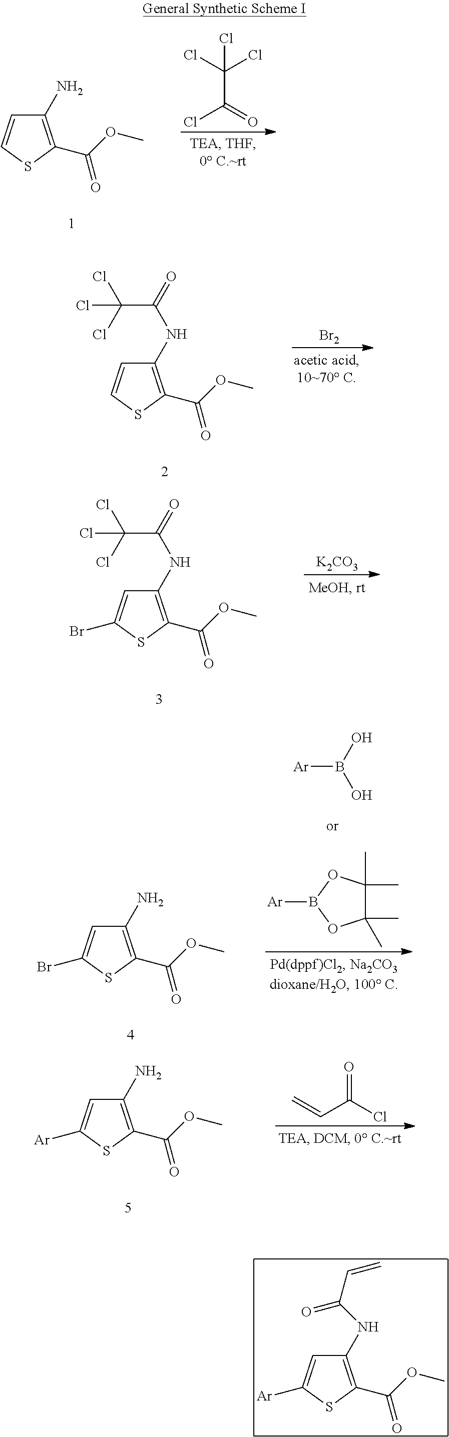

### Methyl 3-(2,2,2-trichloroacetamido)thiophene-2-carboxylate (2)

To a solution of methyl 3-aminothiophene-2-carboxylate (5 g, 31.8 mmol) and triethylamine (4.4 mL, 31.8 mmol) in tetrahydrofuran (40 mL) at 5° C. was added trichloroacetyl chloride (3.5 mL, 31.8 mmol). The reaction was stirred at 5° C. for 30 min. Water (50 mL) was then added and the aqueous phase was extracted with EtOAc. The organic phase was then washed, dried (Na₂SO₄) and the solvent was evaporated to afford methyl 3-(2,2,2-trichloroacetamido)thiophene-2-carboxylate (2). No further purification necessary. Yield 99%. White solid. m/z 302 [M+H]⁺.

### Methyl 5-bromo-3-(2,2,2-trichloroacetamido)thiophene-2-carboxylate (3)

The compound 2 (3.34 g, 11 mmol) obtained in the last step was dissolved in 30 mL of acetic acid, and liquid bromine (5.3 g, 33 mol) was added dropwise at 10° C., and the mixture was stirred for 30 minutes while maintaining the temperature. Raise to 70° C. and heat to stir overnight. After the reaction solution was cooled to room temperature, it was poured into 100 mL of ice water and stirred. The precipitated solid is suction filtered, washed with water, and drained. The filter cake was collected to obtain 3.12 g of a solid compound 3. Yield 74%. m/z 380, 382 [M+H]⁺.

### Methyl 3-amino-5-bromothiophene-2-carboxylate (4)

The compound 3 (3.12 g, 8.2 mmol) obtained in the last step was dissolved in methanol (20 mL), and potassium carbonate (3.4 g, 24.6 mol) was added and stirred at room temperature for 15 hours. Add 100 mL of water, filter the precipitated solids, wash with water, and drain. The filter cake was collected to obtain 1.32 g of solid compound 4.Yield 68%. m/z 236, 238 [M+H]⁺.

### Methyl 3-amino-5-arylthiophene-2-carboxylate (5)

The compound 4 obtained in the last step (1.0 eq), aryl boronic acid or aryl boronic acid pinacol ester (1.2 eq) and Pd(dppf)Cl₂ (0.1 eq) were placed in a vial and dioxane/2M aqueous Na₂CO₃ (v/v=4:1) was added. The resultant reaction mixture was stirred and heated to 100° C. for 3-5 hours. Then it was diluted with EtOAc and filtrated through a Celite pad. The filtrate was concentrated and purified via silica gel flash chromatography to obtain compound 5.

### Methyl 3-acrylamido-5-arylthiophene-2-carboxylate

To a solution of compound 5 obtained in the last step (1.0 eq) and triethylamine (1.5 eq) in DCM at 5° C. was added acryloyl chloride (1.1 eq). The reaction was warmed to rt and stirred for 3-5 hours until LC-MS indicated most of compound 5 was consumed. Then the reaction mixture was purified via silica gel flash chromatography to obtain the desired compound according to different compound 5 as starting material.

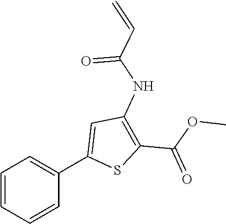 [SMILES: C=CC(=O)Nc1cc(-c2ccccc2)sc1C(=O)OC]

Methyl 3-acrylamido-5-phenylthiophene-2-carboxylate was obtained following General Synthetic Scheme I. m/z 288 [M+H]⁺.

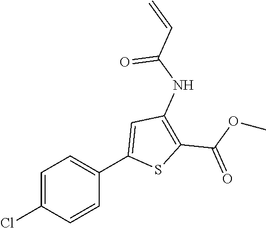 [SMILES: C=CC(=O)Nc1cc(-c2ccc(Cl)cc2)sc1C(=O)OC]

Methyl 3-acrylamido-5-(4-chlorophenyl)thiophene-2-carboxylate was obtained following General Synthetic Scheme I. m/z 322[M+H]⁺.

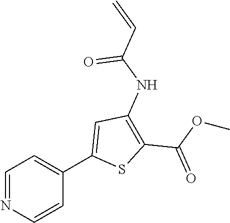 [SMILES: C=CC(=O)Nc1cc(-c2ccncc2)sc1C(=O)OC]

Methyl 3-acrylamido-5-(pyridin-4-yl)thiophene-2-carboxylate was obtained following General Synthetic Scheme I. m/z 289 [M+H]⁺. ¹H-NMR (500 MHz, DMSO-d6) δ (ppm): 10.23 (s, 1H), 8.69-8.64 (m, 2H), 8.51 (s, 1H), 7.74-7.71 (m, 2H), 6.64 (dd, J=17.0, 10.2 Hz, 1H), 6.35 (dd, J=17.0, 1.5 Hz, 1H), 5.90 (dd, J=10.2, 1.5 Hz, 1H), 3.89 (s, 3H).

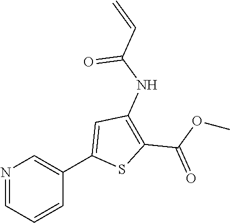 [SMILES: C=CC(=O)Nc1cc(-c2cccnc2)sc1C(=O)OC]

Methyl 3-acrylamido-5-(pyridin-4-yl)thiophene-2-carboxylate was obtained following General Synthetic Scheme I. m/z 289 [M+H]⁺.

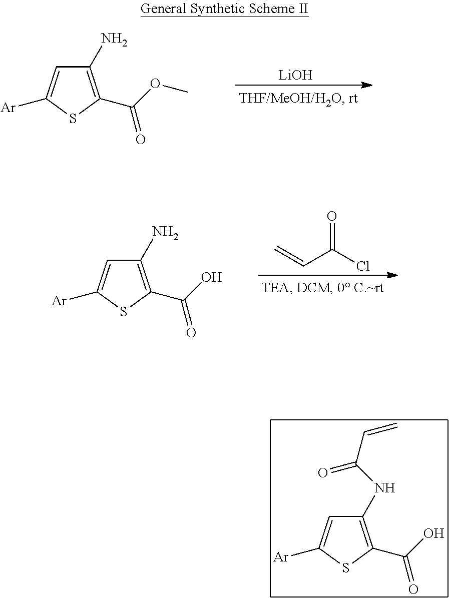

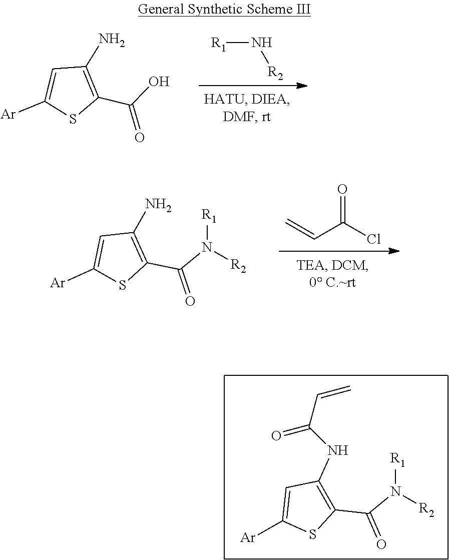

### Synthesis of ethyl 2-amino-5-(pyridin-4-yl)-1H-pyrrole-3-carboxylate

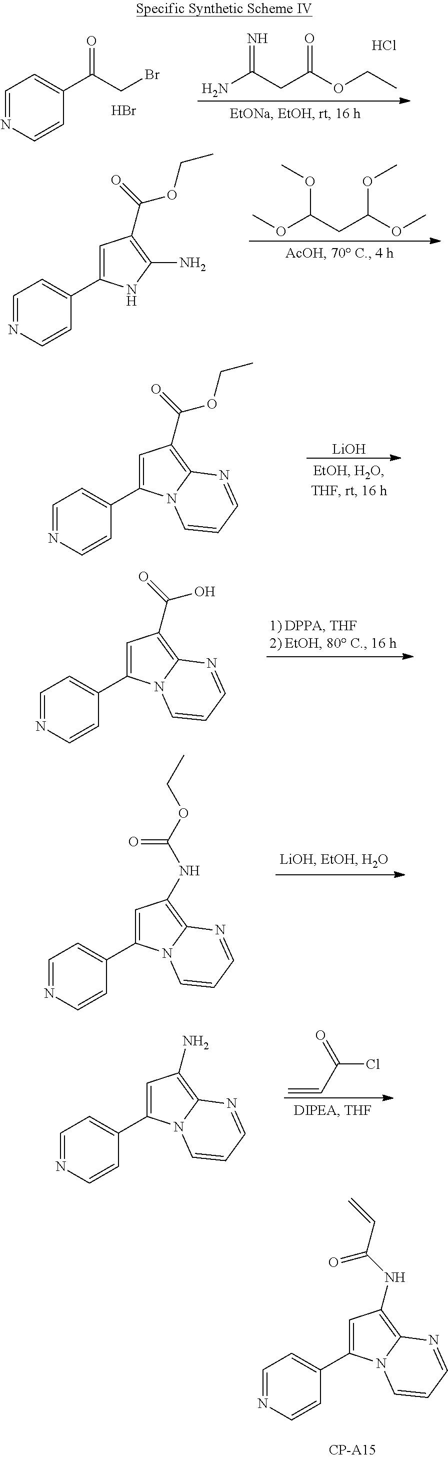

Na (575 mg, 25 mmol) was dissolved in ethanol (70 mL) to get the EtONa solution, and then ethyl 3-amino-3-iminopropanoate hydrochloride (4.15 g, 25 mmol) was added. The mixture was stirred at rt for 30 min. 2-bromo-1-(pyridin-4-yl)ethan-1-one hydrogen bromide (3.5 g, 12.5 mmol) was added in portions, and the resulting mixture was stirred at rt overnight. The mixture was quenched with water (200 mL), concentrated to remove ethanol and extracted with ethyl acetate (100 mL×2). The combined organic layer was dried over anhydrous Na₂SO₄, concentrated and purified with flash column chromatography on silica gel (methanol in DCM, 10% v/v) to give ethyl 2-amino-5-(pyridin-4-yl)-1H-pyrrole-3-carboxylate as colorless oil (2.1 g, yield 52%). LC-MS (ESI) m/z: 232 [M+H]⁺.

### Synthesis of ethyl 6-(pyridin-4-yl)pyrrolo[1,2-a]pyrimidine-8-carboxylate

A mixture of ethyl 2-amino-5-(pyridin-4-yl)-1H-pyrrole-3-carboxylate (1.9 g, 8.22 mmol) and 1,1,3,3-tetramethoxypropane (4 g, 24.64 mmol) in AcOH (30 mL) was stirred at 70° C. for 4 h. The mixture was concentrated in vacuum, the residue was diluted with water (200 mL), adjusted to pH>7 with NaHCO₃ and extracted with ethyl acetate (100 mL×4). The combined organic was dried over anhydrous Na₂SO₄, concentrated and purified with flash column chromatography on silica gel (methanol in DCM, 10% v/v) to afford ethyl 6-(pyridin-4-yl)pyrrolo[1,2-a]pyrimidine-8-carboxylate as a brown solid (1.4 g, yield 58%). LC-MS (ESI) m/z: 268 [M+H]⁺.

### Synthesis of 6-(pyridin-4-yl)pyrrolo[1,2-a]pyrimidine-8-carboxylic acid

A mixture of ethyl 6-(pyridin-4-yl)pyrrolo[1,2-a]pyrimidine-8-carboxylate (1.4 g, 5.24 mmol) and LiOH—H₂O (1.1 g, 26.19 mmol) in THE (30 mL), H₂O (3 mL) and ethanol (30 mL) was stirred at 50° C. overnight. The mixture was adjusted to pH<7 with TFA and concentrated in vacuum, the residue was purified with reverse phase to furnish 6-(pyridin-4-yl)pyrrolo[1,2-a]pyrimidine-8-carboxylic acid as a light yellow solid (1 g, yield 80%). LC-MS (ESI) m/z: 240 [M+H]⁺.

### Synthesis of ethyl (6-(pyridin-4-yl)pyrrolo[1,2-a]pyrimidin-8-yl)carbamate

A mixture of 6-(pyridin-4-yl)pyrrolo[1,2-a]pyrimidine-8-carboxylic acid (320 mg, 1.34 mmol), DPPA (552 mg, 2 mmol) and TEA (404 mg, 4 mmol) in THE (10 mL) was stirred at rt for 6 h. The mixture was concentrated in vacuum, the residue was and dissolved in ethanol (10 mL) and stirred at 80° C. overnight. The mixture was concentrated, the residue was purified with flash column chromatography on silica gel (methanol in DCM, 10% v/v) to yield ethyl (6-(pyridin-4-yl)pyrrolo[1,2-a]pyrimidin-8-yl)carbamate as a brown solid (320 mg, yield 85%). LC-MS (ESI) m/z: 283 [M+H]⁺.

### Synthesis of 6-(pyridin-4-yl)pyrrolo[1,2-a]pyrimidin-8-amine

A mixture of ethyl (6-(pyridin-4-yl)pyrrolo[1,2-a]pyrimidin-8-yl)carbamate (90 mg, 0.319 mmol), LiOH—H₂O (13 mg, 3.19 mmol) in EtOH (5 mL) and H₂O (5 mL) was stirred at 100° C. under microwave irradiation for 1 h. The mixture was concentrated and purified with reverse phase to give 6-(pyridin-4-yl)pyrrolo[1,2-a]pyrimidin-8-amine as a brown solid (50 mg, yield 53%). LC-MS (ESI) m/z: 211 [M+H]⁺.

### Synthesis of N-(6-(pyridin-4-yl)pyrrolo[1,2-a]pyrimidin-8-yl)acrylamide

To a mixture of 6-(pyridin-4-yl)pyrrolo[1,2-a]pyrimidin-8-amine (50 mg, 0.238 mmol) and DIPEA (61 mg, 0.476 mmol) in THE (5 mL) was added dropwise a solution of acryloyl chloride (32 mg, 0.357 mmol) in THE (1 mL) at 0° C. The mixture was stirred at 0° C. for 2 h., quenched with methanol (1 mL), concentrated and purified with prep-HPLC to give N-(6-(pyridin-4-yl)pyrrolo[1,2-a]pyrimidin-8-yl)acrylamide as a brown solid (20 mg, yield 22%). LC-MS (ESI) m/z: 265 [M+H]⁺; ¹H-NMR (DMSO-dδ, 400 MHz): δ (ppm) 5.81-5.84 (m, 1H), 6.41-6.45 (m, 1H), 6.59-6.66 (m, 1H), 7.04-7.06 (m, 1H), 8.19 (d, J=7.2 Hz, 2H), 8.34 (s, 1H), 8.37-8.39 (m, 1H), 8.62 (d, J=6.8 Hz, 2H), 9.18-2.20 (m, 1H).

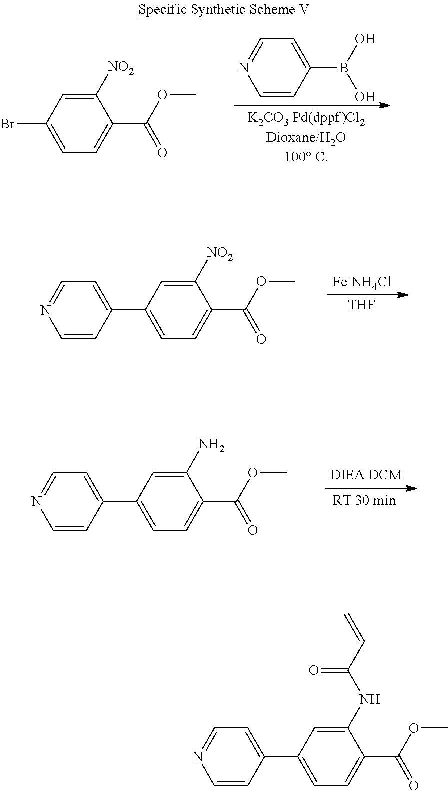

### Synthesis of methyl 2-nitro-4-(pyridin-4-yl)benzoate

A mixture of methyl 4-bromo-2-nitrobenzoate (3.0 g, 11.6 mmol), pyridin-4-ylboronic acid (1.71 g, 13.9 mmol), K₂CO₃ (3.2 g, 23.2 mmol) and Pd(dppf)Cl₂ (848 mg, 1.16 mmol) in dioxane/H₂O (100/10 mL) was stirred at 100° C. under N₂ atmosphere for 12 hours. After cooled down to room temperature the reaction mixture was filtered. The filtrate was concentrated and purified with prep-HPLC to get methyl 2-nitro-4-(pyridin-4-yl)benzoate as a white solid (1.0 g, yield 33%). LC-MS (ESI) m/z: 259[M+H]⁺.

### Synthesis of methyl 2-amino-4-(pyridin-4-yl)benzoate

To a solution of methyl 2-nitro-4-(pyridin-4-yl)benzoate (320 mg, 1.24 mmol) in THF/H₂O (10/1 mL) was added iron powder (694 mg, 12.4 mmol) and NH₄Cl (328 mg, 6.2 mmol). The mixture was stirred at 70° C. for 4 h. and filtered. The filtrate was concentrated and purified with prep-HPLC to get methyl 2-amino-4-(pyridin-4-yl)benzoate as a white solid (100 mg, 35%). LC-MS (ESI) m/z: 229 [M+H]⁺.

### Synthesis of methyl 2-acrylamido-4-(pyridin-4-yl)benzoate

To a solution of methyl 2-amino-4-(pyridin-4-yl)benzoate (100 mg, 0.44 mmol) in DCM (10 mL) was added TEA (88 mg, 0.88 mmol) and acryloyl chloride (46 mg, 0.52 mmol), The mixture was stirred at room temperature for 30 min. and concentrated in vacuum, the resiaue was purified with prep-HIPLC to get methyl 2-acrylamido-4-(pyridin-4-yl)benzoate (A-16) as a white solid (35 mg, yield 28%). LC-MS (ESI) m/z: 283[M+H]⁺; ¹H-NMR (400 MHz, DMSO-d₆): δ (ppm) 3.88 (s, 3H), 5.86-5.89 (m, 1H), 6.28-6.33 (m, 1H), 6.45-6.52 (m, 1H), 7.64-7.67 (m, 1H), 7.71-7.72 (m, 2H), 8.04-8.06 (m, 1H), 8.69-8.71 (m, 3H), 10.84 (s, 1H).

Further exemplary compounds of the invention are shown below:

| | MS m/z | |
|---|---|---|
| Compound | [M + H]⁺ | ¹H NMR |
| 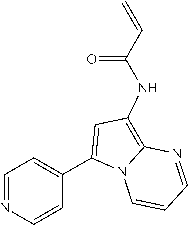 [SMILES: C=CC(=O)Nc1cc(-c2ccncc2)n2cccnc12] | 265 | ¹H-NMR (DMSO-d₆, 400 MHz): δ (ppm) 5.81-5.84 (m, 1H), 6.41-6.45 (m, 1H), 6.59-6.66 (m, 1H), 7.04-7.06 (m, 1H), 8.19 (d, J = 7.2 Hz, 2H), 8.34 (s, 1H), 8.37-8.39 (m, 1H), 8.62 (d, J = 6.8 Hz, 2H), 9.18- 2.20 (m, 1H). |
| 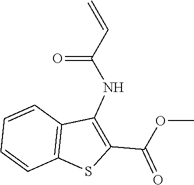 [SMILES: C=CC(=O)Nc1c(C(=O)OC)sc2ccccc12] | 262 | ¹H-NMR (DMSO-d₆, 400 MHz): δ (ppm) 3.82 (s, 3H), 5.82-5.85 (m, 1H), 6.27-6.32 (m, 1H), 6.56-6.63 (m, 1H), 7.46-7.50 (m, 1H), 7.56-7.58 (m, 1H), 7.77-7.79 (m, 1H), 8.02-8.04 (m, 1H), 10.34 (s, 1H). |
| 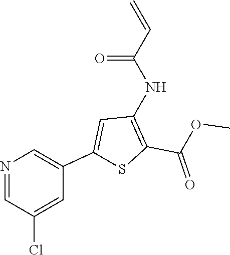 [SMILES: C=CC(=O)Nc1cc(-c2cncc(Cl)c2)sc1C(=O)OC] | 323.04/325.04 | 1H NMR (500 MHZ, DMSO-d₆) δ 10.24 (s, 1H), 8.91 (d, J = 2.0 Hz, 1H), 8.68 (d, J = 2.3 Hz, 1H), 8.46 (s, 1H), 8.36 (t, J = 2.2 Hz, 1H), 6.64 (dd, J = 16.9, 10.2 Hz, 1H), 6.35 (dd, J = 17.0, 1.5 Hz, 1H), 5.91 (dd, J = 10.3, 1.5 Hz, 1H), 3.89 (s, 3H). |
| 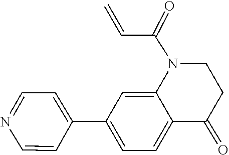 | 279.24 | ¹H NMR (500 MHz, DMSO-d₆) δ 8.84 (s, 2H), 8.04 (d, J = 8.2 Hz, 1H), 7.98 (d, J = 5.3 Hz, 2H), 7.92 (s, 1H), 7.81 (dd, J = 8.2, 1.8 Hz, 1H), 6.82 (dd, J = 16.7, 10.4 Hz, 1H), 6.33 (dd, J = 16.6, 2.0 Hz, 1H), 5.88 (dd, J = 10.4, 2.1 Hz, 1H), 4.29 (t, J = 6.2 Hz, 2H), 2.86 (t, J = 6.2 Hz, 2H). |
| 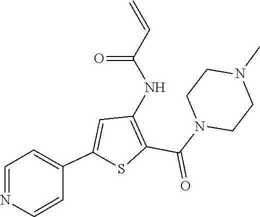 [SMILES: C=CC(=O)Nc1cc(-c2ccncc2)sc1C(=O)N1CCN(C)CC1] | 357.15 | |
| 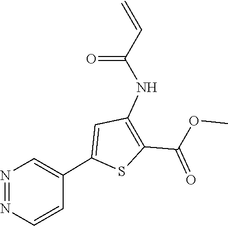 [SMILES: C=CC(=O)Nc1cc(-c2ccnnc2)sc1C(=O)OC] | 236.14 | ¹H NMR (500 MHz, DMSO-d₆) δ 10.25 (s, 1H), 9.65 (dd, J = 2.6, 1.3 Hz, 1H), 9.31 (dd, J = 5.5, 1.2 Hz, 1H), 8.65 (s, 1H), 8.05 (dd, J = 5.5, 2.5 Hz, 1H), 6.66 (dd, J = 17.0, 10.2 Hz, 1H), 6.36 (dd, J = 17.0, 1.5 Hz, 1H), 5.92 (dd, J = 10.4, 1.5 Hz, 1H), 3.91 (s, 3H). |
| 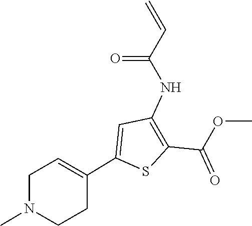 | 307.14 | ¹H NMR (500 MHz, DMSO-d₆) δ 10.19 (s, 1H), 7.95 (s, 1H), 6.58 (dd, J = 16.9, 10.3 Hz, 1H), 6.39 − 6.34 (m, 1H), 6.31 (dd, J = 16.9, 1.5 Hz, 1H), 5.88 (dd, J = 10.3, 1.5 Hz, 1H), 3.84 (s, 3H), 3.03 (q, J = 3.0 Hz, 2H), 2.56 (t, J = 5.7 Hz, 2H), 2.48 − 2.45 (m, 2H), 2.28 (s, 3H). |
| 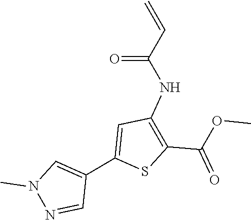 | 292.14 | ¹H NMR (500 MHz, DMSO-d₆) δ 10.23 (s, 1H), 8.29 (s, 1H), 8.06 (s, 1H), 7.88 (s, 1H), 6.60 (dd, J = 17.0, 10.3 Hz, 1H), 6.33 (dd, J = 17.0, 1.5 Hz, 1H), 5.89 (dd, J = 10.3, 1.5 Hz, 1H), 3.88 (s, 3H), 3.85 (s, 3H). |
| 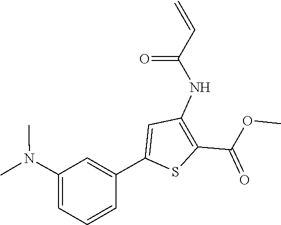 | 331.14 | ¹H NMR (500 MHZ, DMSO-d₆) δ 10.23 (s, 1H), 8.29 (s, 1H), 7.30 (t, J = 7.9 Hz, 1H), 7.02 (dd, J = 7.3, 1.7 Hz, 1H), 6.99 - 6.93 (m, 1H), 6.85 (dd, J = 8.5, 2.5 Hz, 1H), 6.62 (dd, J = 16.9, 10.3 Hz, 1H), 6.34 (dd, J = 16.9, 1.5 Hz, 1H), 5.90 (dd, J = 10.4, 1.5 Hz, 1H), 3.87 (s, 3H), 2.98 (s, 6H). |
| 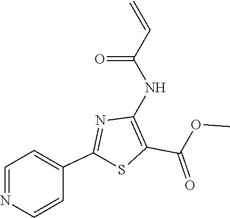 [SMILES: C=CC(=O)Nc1nc(-c2ccncc2)sc1C(=O)OC] | 290.14 | ¹H NMR (500 MHZ, DMSO-d₆) δ 10.85 (s, 1H), 8.82 (s, 2H), 8.01 - 7.97 (m, 2H), 6.56 (dd, J = 17.1, 10.3 Hz, 1H), 6.33 (dd, J = 17.1, 1.7 Hz, 1H), 5.87 (dd, J = 10.3, 1.8 Hz, 1H), 3.82 (s, 3H). |
| 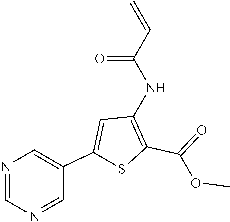 [SMILES: C=CC(=O)Nc1cc(-c2cncnc2)sc1C(=O)OC] | 290.14 | ¹H NMR (500 MHz, DMSO-d₆) δ 10.25 (s, 1H), 9.22 (d, J = 10.7 Hz, 2H), 8.48 (s, 1H), 6.64 (dd, J = 17.0, 10.3 Hz, 1H), 6.35 (dd, J = 17.0, 1.5 Hz, 1H), 5.91 (dd, J = 10.3, 1.5 Hz, 1H), 3.90 (s, 3H). |
| 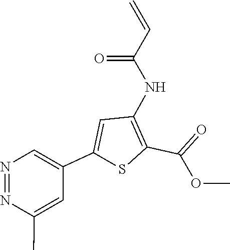 [SMILES: C=CC(=O)Nc1cc(-c2cnnc(C)c2)sc1C(=O)OC] | 304.14 | ¹H NMR (500 MHZ, DMSO-d₆) δ 10.24 (s, 1H), 9.45 (d, J = 2.4 Hz, 1H), 8.63 (s, 1H), 7.96 (d, J = 2.4 Hz, 1H), 6.65 (dd, J = 17.0, 10.2 Hz, 1H), 6.36 (dd, J = 16.8, 1.5 Hz, 1H), 5.91 (dd, J = 10.3, 1.5 Hz, 1H), 3.90 (s, 3H), 2.69 (s, 3H). |
| 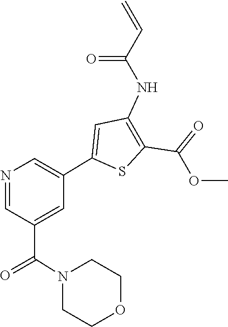 [SMILES: C=CC(=O)Nc1cc(-c2cncc(C(=O)N3CCOCC3)c2)sc1C(=O)OC] | 402.20 | ¹H NMR (500 MHZ, DMSO-d₆) δ 10.25 (s, 1H), 9.02 (d, J = 2.3 Hz, 1H), 8.66 (d, J = 1.9 Hz, 1H), 8.46 (s, 1H), 8.22 (t, J = 2.1 Hz, 1H), 6.64 (dd, J = 17.0, 10.2 Hz, 1H), 6.35 (dd, J = 16.9, 1.5 Hz, 1H), 5.91 (dd, J = 10.1, 1.5 Hz, 1H), 3.89 (s, 3H), 3.74- 3.52 (m, 4H), 3.44-3.26 (m, 4H). |
| 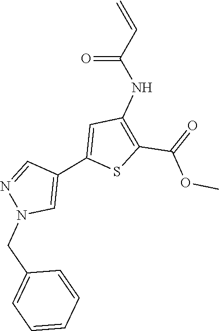 [SMILES: C=CC(=O)Nc1cc(-c2cnn(Cc3ccccc3)c2)sc1C(=O)OC] | 368.15 | ¹H NMR (500 MHZ, DMSO-d₆) δ 10.23 (s, 1H), 8.47 (s, 1H), 8.08 (s, 1H), 7.93 (s, 1H), 7.37 (dd, J = 8.0, 6.5 Hz, 2H), 7.34- 7.27 (m, 3H), 6.59 (dd, J = 17.0, 10.3 Hz, 1H), 6.32 (dd, J = 17.0, 1.5 Hz, 1H), 5.89 (dd, J = 10.3, 1.5 Hz, 1H), 5.36 (s, 2H), 3.85 (s, 3H). |
| 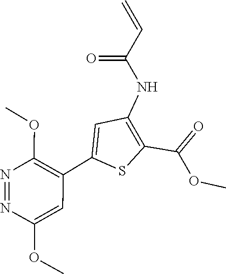 [SMILES: C=CC(=O)Nc1cc(-c2cc(OC)nnc2OC)sc1C(=O)OC] | 350.16 | ¹H NMR (500 MHz, DMSO-d₆) δ 10.19 (s, 1H), 8.67 (s, 1H), 7.62 (s, 1H), 6.63 (dd, J = 17.0, 10.2 Hz, 1H), 6.34 (dd, J = 16.9, 1.5 Hz, 1H), 5.90 (dd, J = 10.2, 1.5 Hz, 1H), 4.13 (s, 3H), 4.00 (s, 3H), 3.89 (s, 3H). |
| 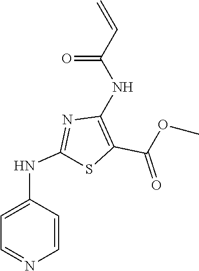 [SMILES: C=CC(=O)Nc1nc(Nc2ccncc2)sc1C(=O)OC] | 305.32 | ¹H NMR (500 MHZ, DMSO-d₆) δ 12.09 (s, 1H), 9.30- 9.19 (m, 2H), 8.26 (d, J = 7.0 Hz, 2H), 7.45 (s, 2H), 6.59 − 6.51 (m, 2H), 6.11 (dd, J = 8.2, 3.4 Hz, 1H), 3.82 (s, 3H). |
| 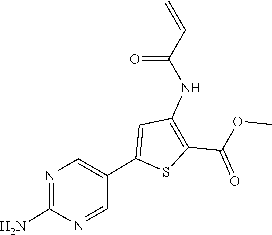 [SMILES: C=CC(=O)Nc1cc(-c2cnc(N)nc2)sc1C(=O)OC] | 305.14 | ¹H NMR (500 MHZ, DMSO-d₆) δ 10.24 (s, 1H), 8.63 (s, 2H), 8.19 (s, 1H), 7.20 (s, 2H), 6.61 (dd, J = 17.0, 10.3 Hz, 1H), 6.33 (dd, J = 16.9, 1.5 Hz, 1H), 5.89 (dd, J = 10.2, 1.5 Hz, 1H), 3.86 (s, 3H). |
| 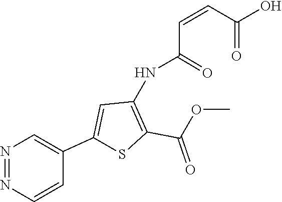 [SMILES: COC(=O)c1sc(-c2ccnnc2)cc1NC(=O)/C=C\C(=O)O] | 333.34 | ¹H NMR (500 MHZ, DMSO-d₆) δ 13.00 (s, 1H), 10.33 (s, 1H), 9.65 (dd, J = 2.5, 1.2 Hz, 1H), 9.31 (dd, J = 5.4, 1.3 Hz, 1H), 8.60 (s, 1H), 8.06 (dd, J = 5.5, 2.6 Hz, 1H), 6.67 (d, J = 12.0 Hz, 1H), 6.45 (d, J = 12.0 Hz, 1H), 3.90 (s, 3H). |
| | 312.04/314.09 | ¹H NMR (500 MHZ, DMSO-d₆) δ 10.75 (s, 1H), 9.66 (dd, J = 2.5, 1.1 Hz, 1H), 9.32 (d, J = 5.5 Hz, 1H), 8.62 (s, 1H), 8.07 (dd, J = 5.5, 2.5 Hz, 1H), 4.58 (s, 2H), 3.92 (s, 3H). |
| | 306.09 | |
| | 284.34 | ¹H NMR (500 MHZ, DMSO-d₆) δ 10.81 (s, (dd, J = 5.4, 1.2 Hz, 1H), 8.70 (d, J = 1.9 Hz, 1H), 8.07 (d, J = 8.2 Hz, 1H), 8.02 (dd, J = 5.4, 2.6 Hz, 1H), 7.78 (dd, J = 8.2, 1.9 Hz, 1H), 6.50 (dd, J = 17.1, 10.3 Hz, 1H), 6.32 (dd, J = 17.1, 1.5 Hz, 1H), 5.89 (dd, J = 10.2, 1.5 Hz, 1H), 3.89 (s, 3H). |
| | 333.36 | ¹H NMR (500 MHZ, DMSO-d₆) δ 11.03 (s, 1H), 9.66 - 9.63 (m, 1H), 9.31 (d, J = 5.6 Hz, 1H), 8.64 (s, 1H), 8.06 (dd, J = 5.5, 2.5 Hz, 1H), 7.93 (s, 1H), 7.48 (s, 1H), 6.49 - 6.30 (m, 2H), 3.89 (s, 3H). |

### Example 2: Method for Assessing UCP1-Dependent Respiration in Primary Brown Adipocytes

Primary brown adipocyte preparation and differentiation. Interscapular brown adipose stromal vascular fraction was obtained from 2- to 6-day-old pups as described in PMID: 30022159. Interscapular brown adipose was dissected, washed in PBS, minced, and digested for 45 min at 37° C. in PBS containing 1.5 mg ml⁻¹ collagenase B, 123 mM NaCl, 5 mM KCl, 1.3 mM CaCl₂, 5 mM glucose, 100 mM HEPES, and 4% essentially fatty-acid-free BSA. Tissue suspension was filtered through a 40 m cell strainer and centrifuged at 600 g for 5 min to pellet the SVF. The cell pellet was resuspended in adipocyte culture medium and plated. Cells were maintained at 37° C. in 10% CO₂. Primary brown pre-adipocytes were counted and plated in the evening, 12 h before differentiation at 15,000 cells per well of a seahorse plate. Pre-adipocyte plating was scaled according to surface area. The following morning, brown pre-adipocytes were induced to differentiate for 2 days with an adipogenic cocktail (1 μM rosiglitazone, 0.5 mM IBMX, 5 μM dexamethasone, 0.114 μg ml-1 insulin, 1 nM T3, and 125 μM Indomethacin) in adipocyte culture medium. Two days after induction, cells were re-fed every 48 h with adipocyte culture medium containing 1 μM rosiglitazone and 0.5 μg ml⁻¹ insulin. Cells were fully differentiated by day 6 after induction.

Direct measure of UCP1-dependent respiration in brown adipocytes. Cellular oxygen consumption and UCP1-rependent respiration of primary brown adipocytes was using a Seahorse XF24 Extracellular Flux Analyzer as described in PMID 30022159. Adipocytes were plated and differentiated in XF24 V7 cell culture microplates. At day 6 differentiation, prior to analysis adipocyte culture medium was changed to respiration medium consisting of DMEM lacking NaHCO₃ (Sigma), NaCl (1.85 g/L), phenol red (3 mg/L), 2% fatty acid free BSA, and sodium pyruvate (1 mM), pH 7.4. Basal respiration was determined to be the oxygen consumption in the presence of substrate (1 mM sodium pyruvate) alone. Following determination of basal respiration, MYF compounds at 100 uM or vehicle was added to the cells and the respiration response measured immediately. To determine UCP1-dependent leak respiration in these cells, oligomycin (4.16 μM) was added. Maximal respiration capacity was determined following addition of DNP (2 mM). Rotenone (3 μM) and antimycin (3 μM) were used to abolish mitochondrial respiration.

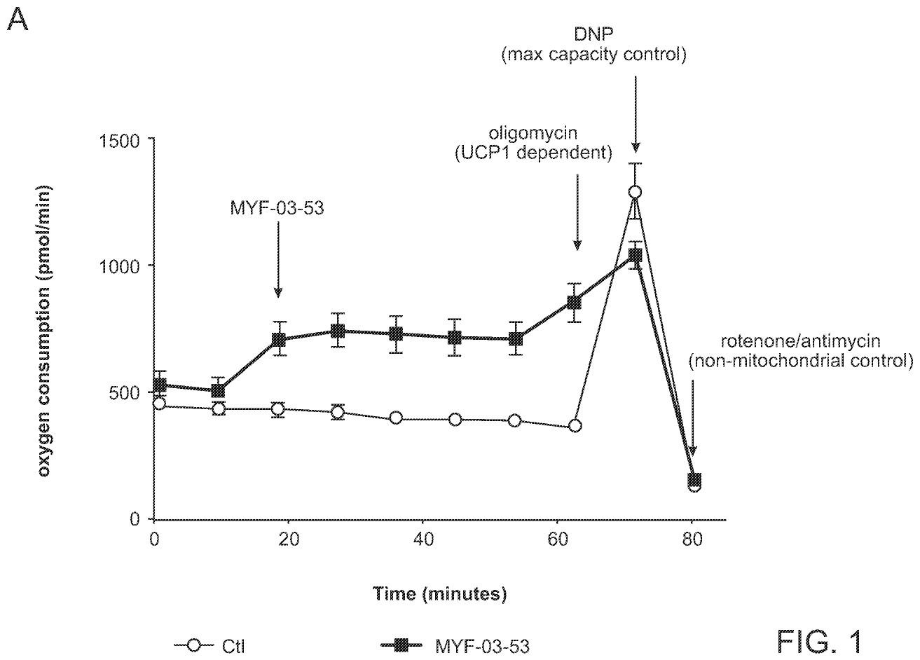

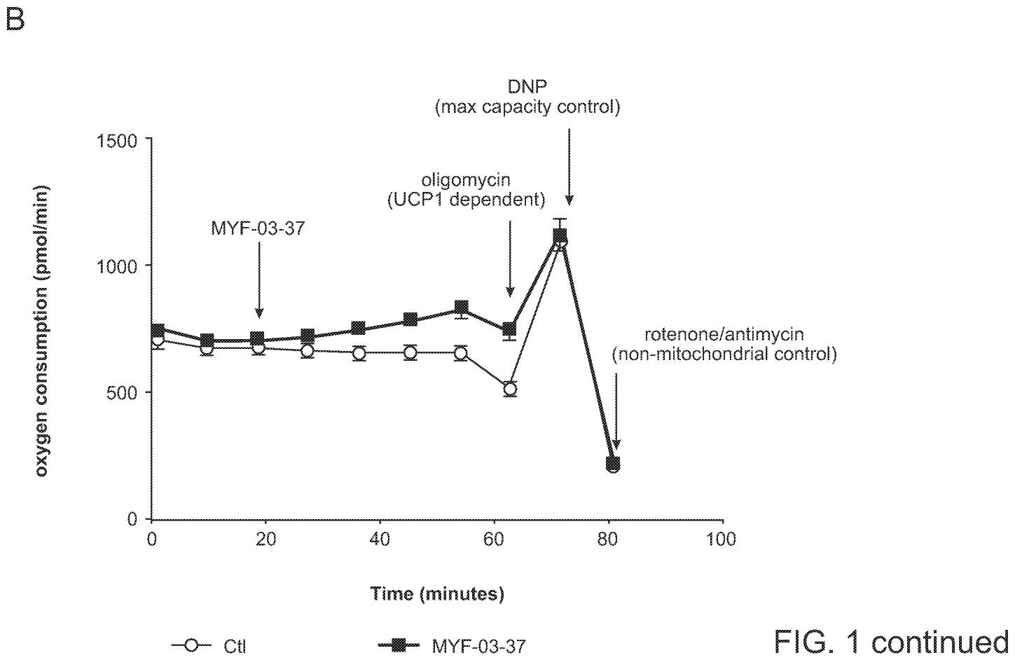

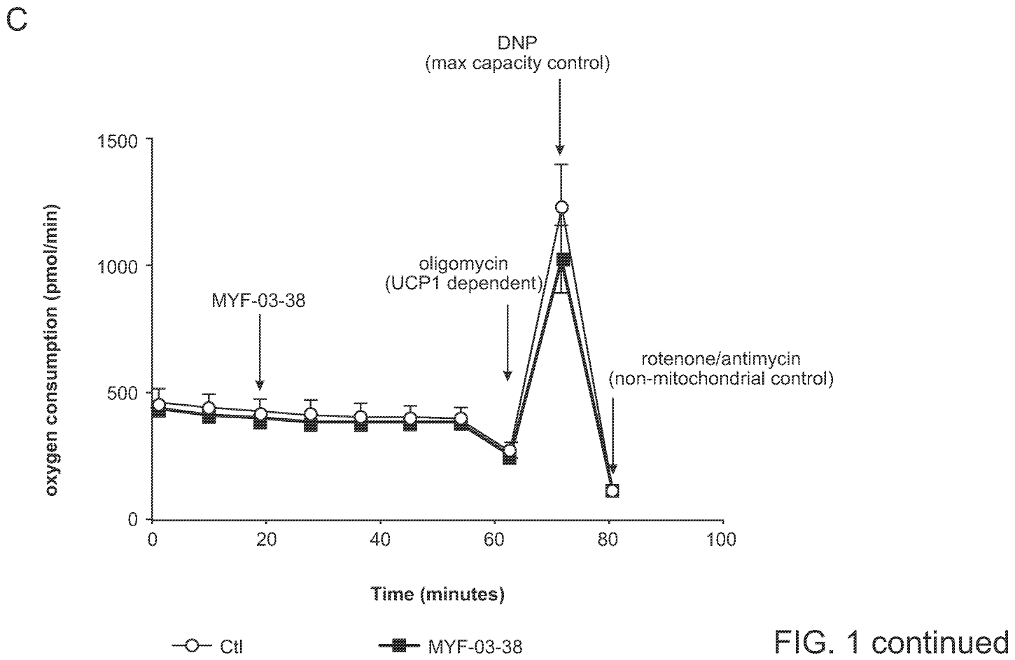

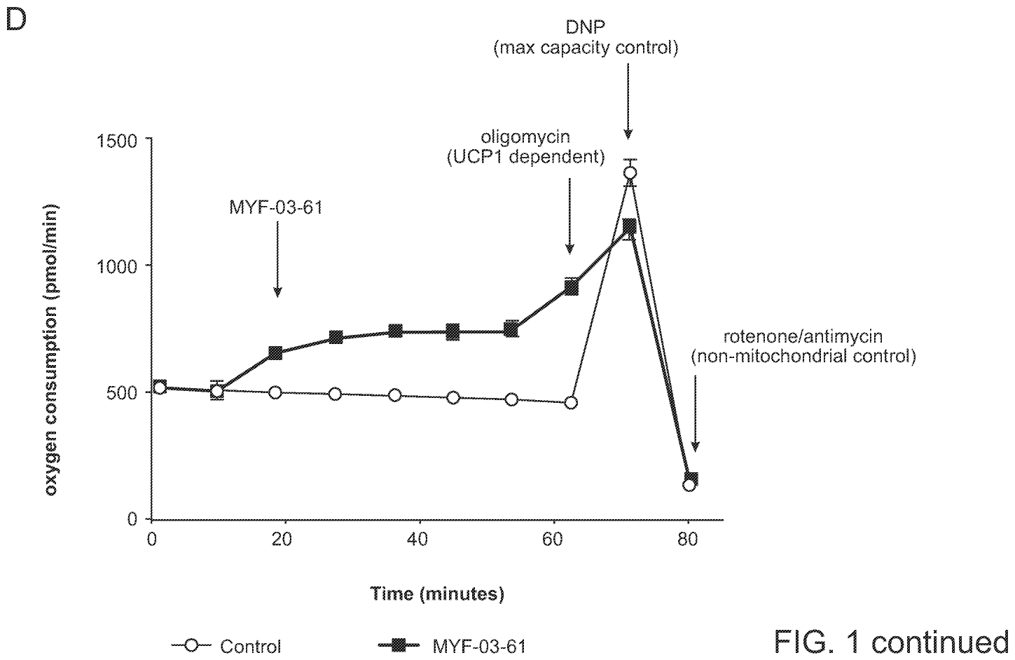

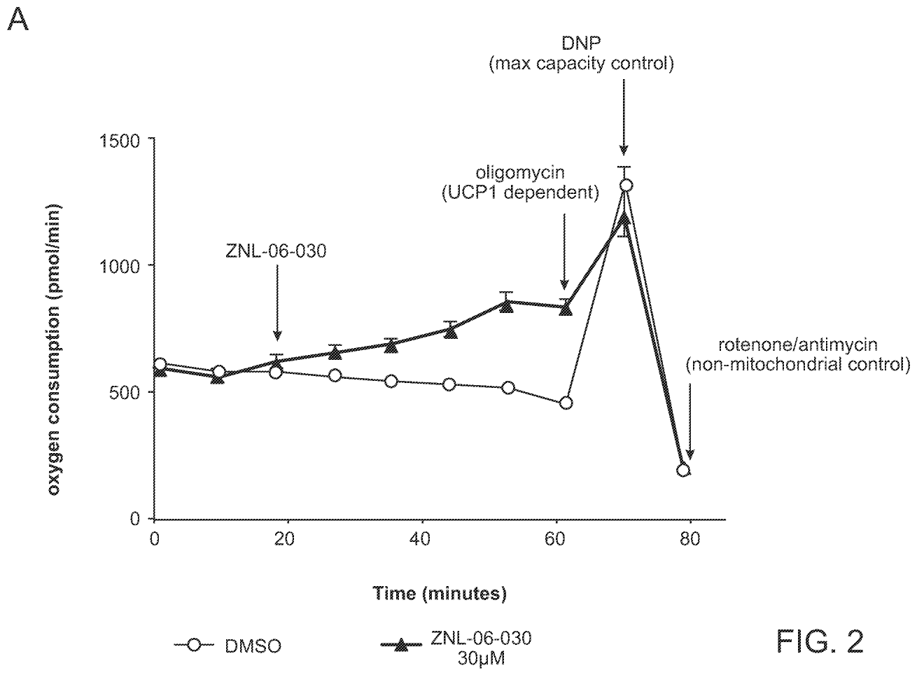

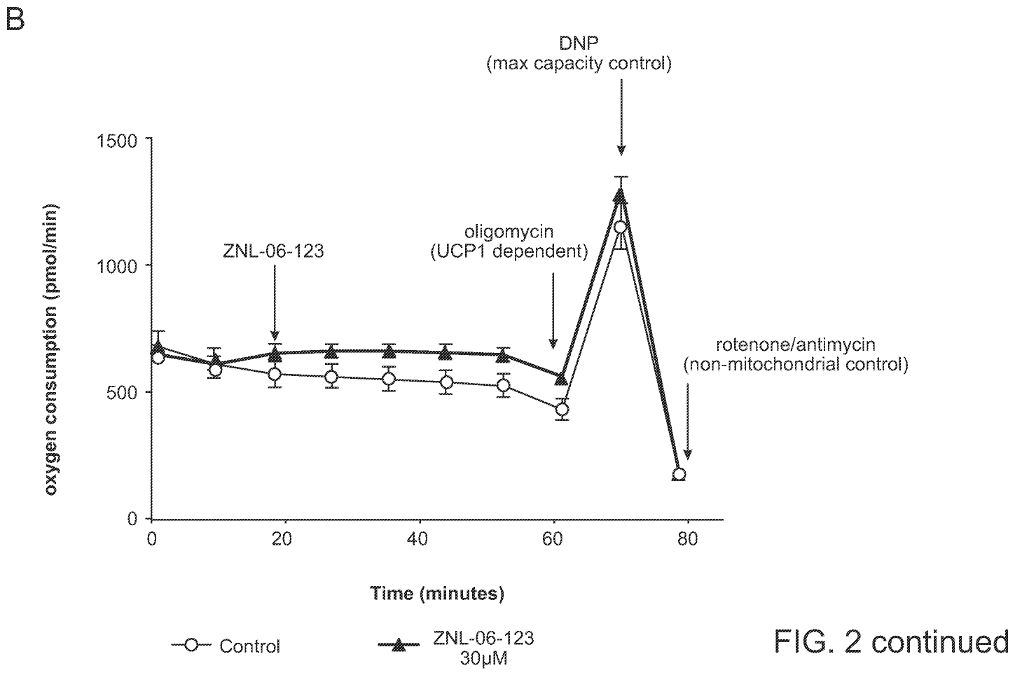

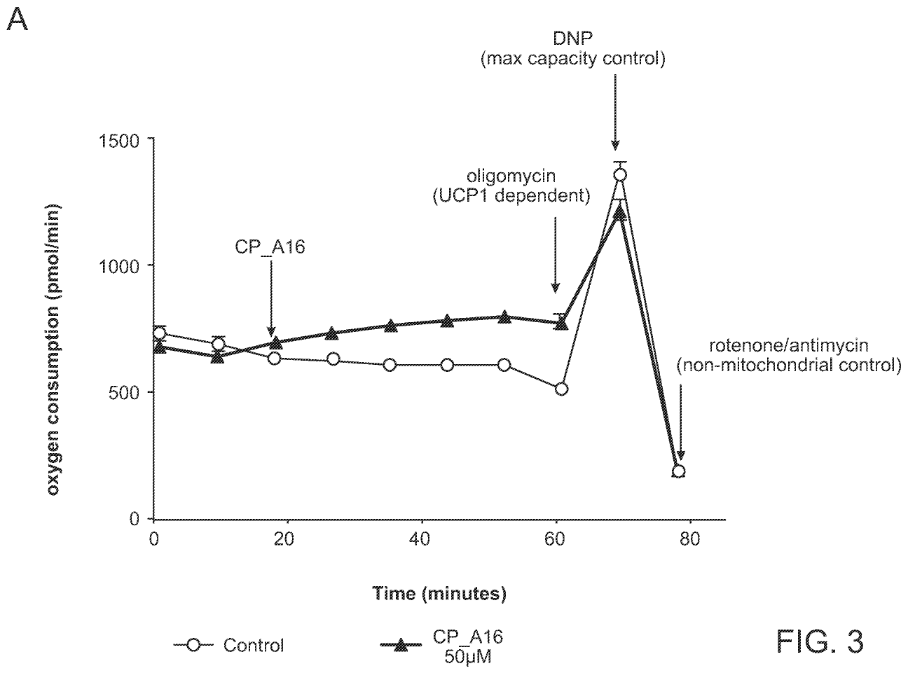

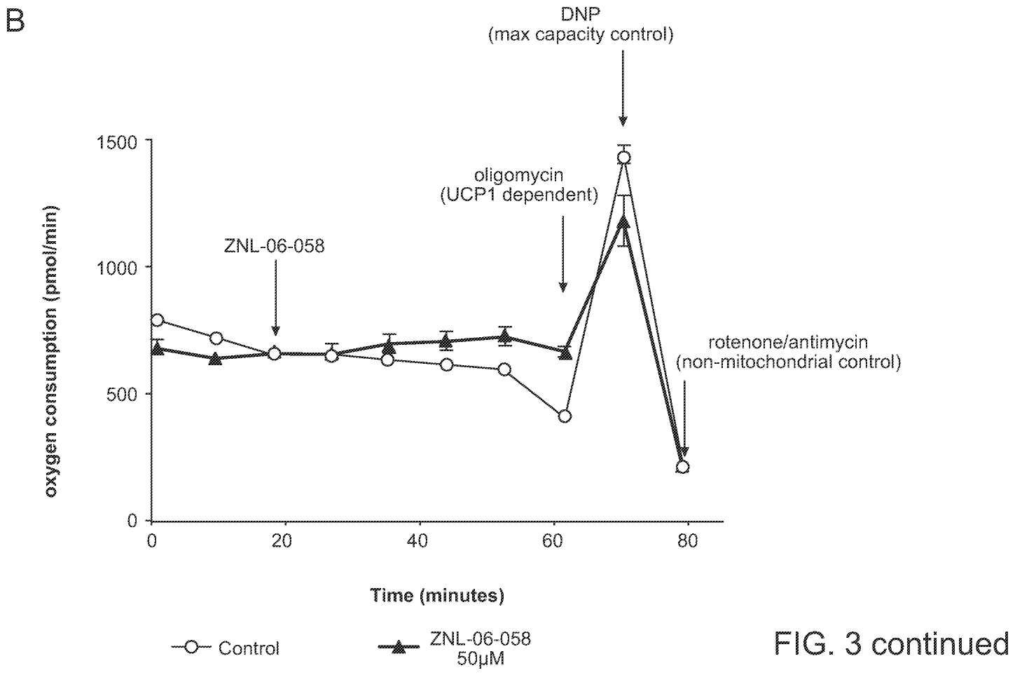

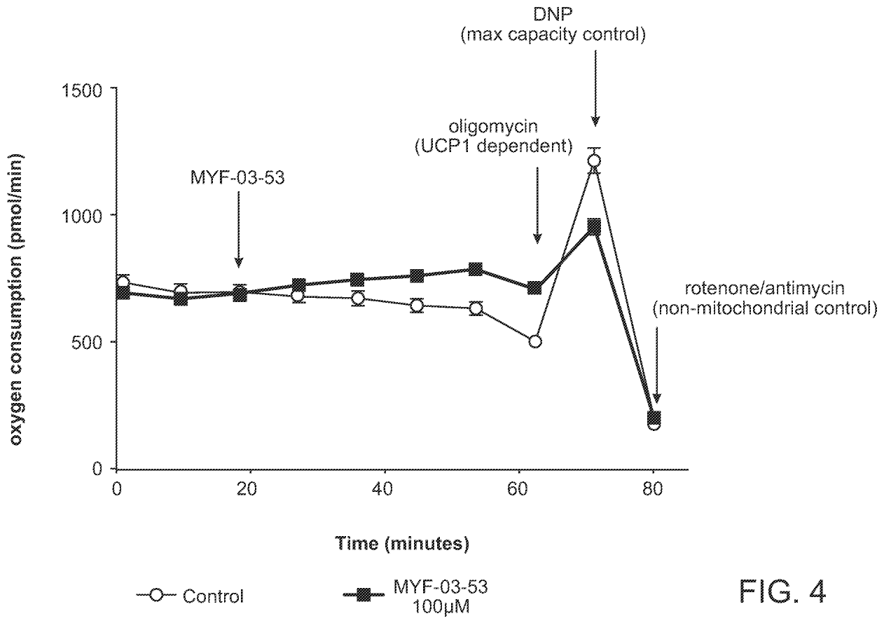

Results are shown in FIGS. 1-4.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

We claim:

1. A compound, or a pharmaceutically acceptable salt thereof, having the structure of formula (I); 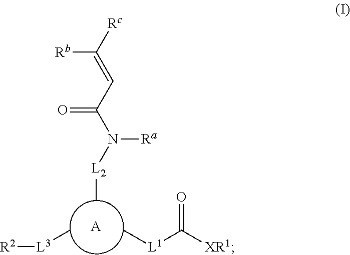
wherein: 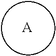
represents a heteroaryl or aryl ring;
L¹ represents —CH₂— or a bond;
L² represents —CH₂— or a bond;
R^{a} represents H or alkyl;
R^{b} represents H, alkyl, —C(O)OH, or —C(O)NH₂;
R^{c} represents H, alkyl, —C(O)OH, or —C(O)NH₂;
X represents —O—, —NH—, or —N(alkyl)-;
R¹ represents H or optionally substituted alkyl, cycloalkyl, aryl, or heteroaryl;
or, XR¹, taken together, represent optionally substituted heterocycloalkyl, wherein the optionally substituted heterocycloalkyl is attached to the carbonyl group through a nitrogen atom;
L³ represents —NH— or a bond; and
R² represents optionally substituted aryl, heteroaryl, or heterocycloalkenyl.

2. The compound of claim 1, wherein 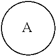
represents a heteroaryl ring.

3. The compound of claim 1 or 2, wherein 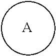
represents a thiazole ring or a thiophene ring.

4. The compound of any one of claims 1-3, wherein 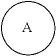
represents a thiophene ring.

5. The compound of any one of claims 1-4, having the structure of formula (Ib):

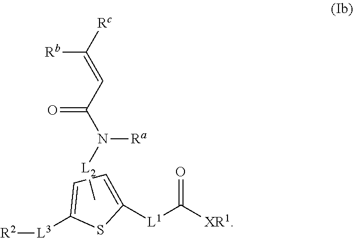

6. The compound of any one of claims 1-5, having the structure of formula (Ic):

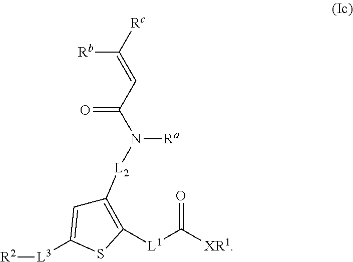

7. The compound of any one of claims 1-3, having the structure of formula (Id):

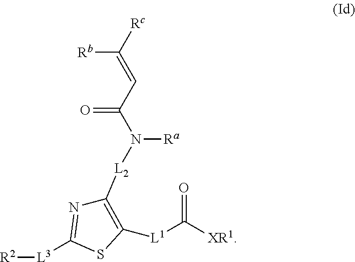

8. The compound of claim 1, wherein 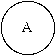
represents a phenyl ring.

9. The compound of claim 1, having the structure of formula (Ia):

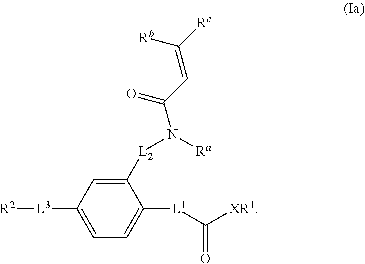

10. The compound of any one of claims 1-9, wherein L¹ represents —CH₂—.

11. The compound of any one of claims 1-9, wherein L¹ represents a bond.

12. The compound of any one of claims 1-11, wherein L² represents —CH₂—.

13. The compound of any one of claims 1-11, wherein L² represents a bond.

14. The compound of any one of claims 1-13, wherein R^{a} represents H.

15. The compound of any one of claims 1-13, wherein R^{a} represents methyl.

16. The compound of any one of claims 1-15, wherein X represents —O—.

17. The compound of any one of claims 1-15, wherein X represents —NH—.

18. The compound of any one of claims 1-17, wherein R¹ represents H.

19. The compound of any one of claims 1-17, wherein R¹ represents optionally substituted alkyl.

20. The compound of claim 19, wherein R¹ represents methyl.

21. The compound of any one of claims 1-17, wherein R¹ represents optionally substituted cycloalkyl.

22. The compound of claim 21, wherein R¹ represents cyclopropyl.

23. The compound of any one of claims 1-22, wherein R² represents optionally substituted aryl or heteroaryl.

24. The compound of any one of claims 1-23, wherein R² is optionally substituted aryl.

25. The compound of claim 24, wherein R² is phenyl and is optionally substituted with one or more substituents selected from halo, alkyl, haloalkyl, alkoxy, haloalkoxy, cyano, nitro, and (alkyl)sulfonyl.

26. The compound of any one of claims 1-23, wherein R² is optionally substituted heteroaryl.

27. The compound of claim 26, wherein R² is pyridyl, pyridazinyl, pyrimidinyl, or pyrazinyl, optionally substituted with one or more substituents selected from the group consisting of halo, alkyl, haloalkyl, alkoxy, haloalkoxy, cyano, nitro, and (alkyl)sulfonyl.

28. The compound of any one of claims 1-27, wherein L³ represents a bond.

29. The compound of any one of claims 1-28, wherein R^{b} represents H.

30. The compound of any one of claims 1-29, wherein R^{c} represents H.

31. The compound of claim 1, having the structure of formula (Ie); 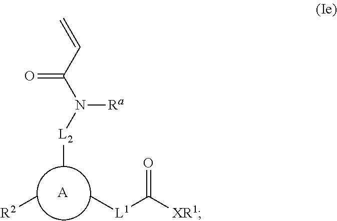
wherein: 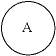
represents a heteroaryl or aryl ring;
L¹ represents —CH₂— or a bond;
L² represents —CH₂— or a bond;
R^{a} represents H or alkyl;
X represents —O—, —NH—, or —N(alkyl)-;
R¹ represents H or optionally substituted alkyl, cycloalkyl, aryl, or heteroaryl;
R² represents optionally substituted aryl, heteroaryl.

32. The compound of claim 1, selected from the following table:

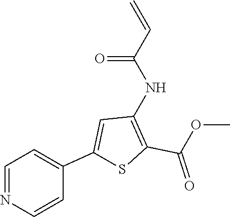 [SMILES: C=CC(=O)Nc1cc(-c2ccncc2)sc1C(=O)OC]

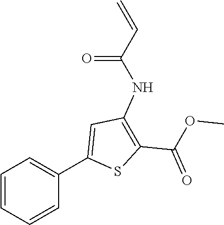 [SMILES: C=CC(=O)Nc1cc(-c2ccccc2)sc1C(=O)OC]

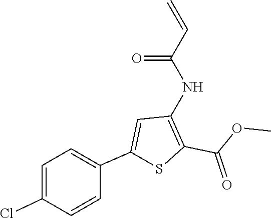 [SMILES: C=CC(=O)Nc1cc(-c2ccc(Cl)cc2)sc1C(=O)OC]

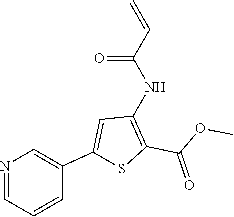 [SMILES: C=CC(=O)Nc1cc(-c2cccnc2)sc1C(=O)OC]

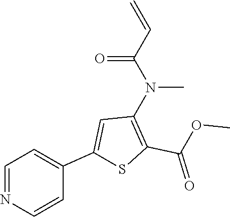 [SMILES: C=CC(=O)N(C)c1cc(-c2ccncc2)sc1C(=O)OC]

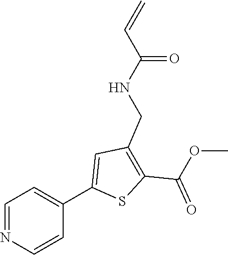 [SMILES: C=CC(=O)NCc1cc(-c2ccncc2)sc1C(=O)OC]

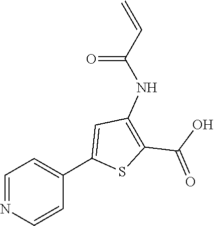 [SMILES: C=CC(=O)Nc1cc(-c2ccncc2)sc1C(=O)O]

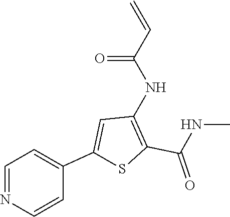 [SMILES: C=CC(=O)Nc1cc(-c2ccncc2)sc1C(=O)NC]

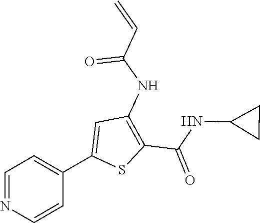 [SMILES: C=CC(=O)Nc1cc(-c2ccncc2)sc1C(=O)NC1CC1]

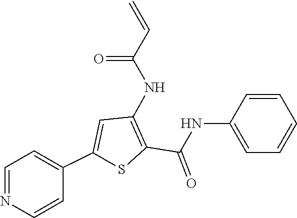 [SMILES: C=CC(=O)Nc1cc(-c2ccncc2)sc1C(=O)Nc1ccccc1]

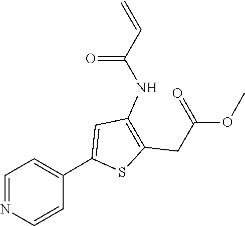 [SMILES: C=CC(=O)Nc1cc(-c2ccncc2)sc1CC(=O)OC]

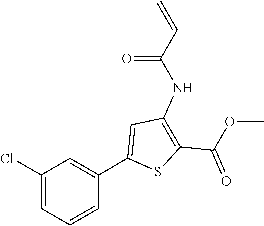 [SMILES: C=CC(=O)Nc1cc(-c2cccc(Cl)c2)sc1C(=O)OC]

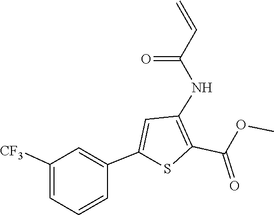 [SMILES: C=CC(=O)Nc1cc(-c2cccc(C(F)(F)F)c2)sc1C(=O)OC]

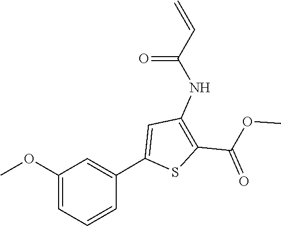

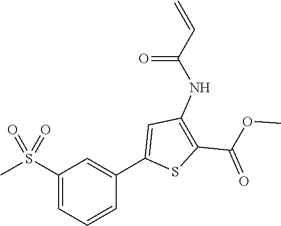

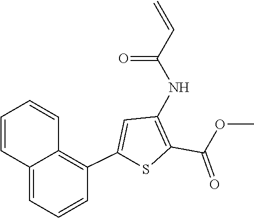 [SMILES: C=CC(=O)Nc1cc(-c2cccc3ccccc23)sc1C(=O)OC]

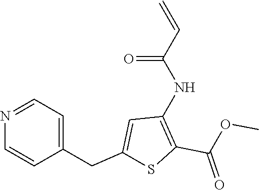 [SMILES: C=CC(=O)Nc1cc(Cc2ccncc2)sc1C(=O)OC]

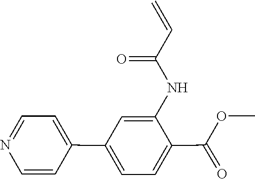 [SMILES: C=CC(=O)Nc1cc(-c2ccncc2)ccc1C(=O)OC]

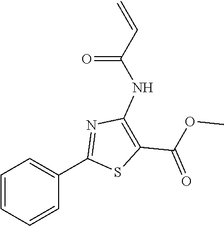 [SMILES: C=CC(=O)Nc1nc(-c2ccccc2)sc1C(=O)OC]

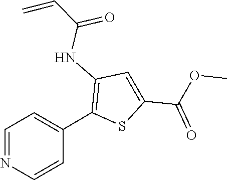 [SMILES: C=CC(=O)Nc1cc(C(=O)OC)sc1-c1ccncc1]

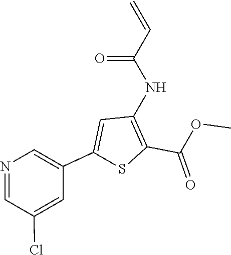 [SMILES: C=CC(=O)Nc1cc(-c2cncc(Cl)c2)sc1C(=O)OC]

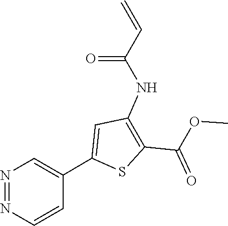 [SMILES: C=CC(=O)Nc1cc(-c2ccnnc2)sc1C(=O)OC]

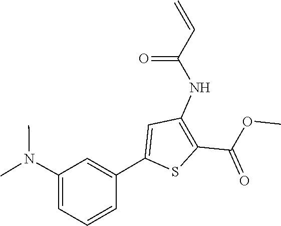

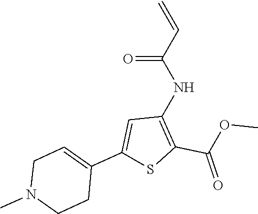

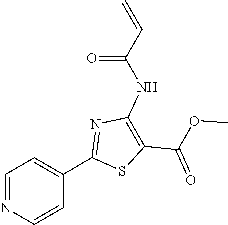 [SMILES: C=CC(=O)Nc1nc(-c2ccncc2)sc1C(=O)OC]

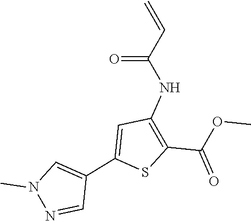

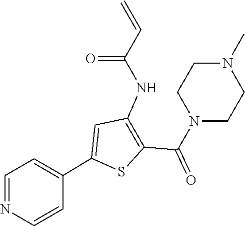 [SMILES: C=CC(=O)Nc1cc(-c2ccncc2)sc1C(=O)N1CCN(C)CC1]

33. A pharmaceutical composition, comprising a compound of any one of claims 1-32, or a pharmaceutically acceptable salt thereof, and a pharmaceutically acceptable excipient.

34. A method of activating uncoupling protein 1 (UCP1) in a cell, comprising contacting the cell with a compound of any one of claims 1-32.

35. A method of treating obesity, the method comprising administering to a subject a therapeutically effective amount of a compound of any one of claims 1-32.

36. A method of lowering the weight of a subject, the method comprising administering to the subject a therapeutically effective amount of a compound of any one of claims 1-32.

37. A method of stimulating calorie burning in a subject, the method comprising administering to the subject a therapeutically effective amount of a compound of any one of claims 1-32.

38. A method of treating a metabolic disorder, the method comprising administering to a subject a therapeutically effective amount of a compound of any one of claims 1-32.

39. The method of claim 33, wherein the metabolic disorder is diabetes.

40. The method of claim 33, wherein the metabolic disorder is nonalcoholic steatohepatitis.
